(19) United States
(12) Patent Application Publication
(10) Pub. No.: US 2013/0261405 A1
(43) Pub. Date: Oct. 3, 2013

(54) APPARATUS AND METHOD FOR MEASURING BIOLOGICAL SIGNAL

(71) Applicant: SAMSUNG ELECTRONICS CO., LTD., Gyeonggi-do (KR)

(72) Inventors: Sang-Hun Lee, Gyeonggi-do (KR); Jae-Geol Cho, Gyeonggi-do (KR)

(73) Assignee: Samsung Electronics Co., Ltd., Gyeonggi-do (KR)

(21) Appl. No.: 13/855,317

(22) Filed: Apr. 2, 2013

(30) Foreign Application Priority Data
Apr. 2, 2012 (KR) 10-2012-0033790

(51) Int. Cl. A61B 5/00; A61B 5/0205; A61B 5/01; A61B 5/0402; A61B 5/145

(52) U.S. Cl. CPC A61B 5/681; A61B 5/0402; A61B 5/0082; A61B 5/14542; A61B 5/14532; A61B 5/01; A61B 5/02055; A61B 5/0002; A61B 5/742
USPC 600/301; 600/300; 600/509; 600/479; 600/508; 600/485; 600/364; 600/529; 600/365; 600/549; 600/484; 600/483

(57) ABSTRACT

An apparatus and method for easily and accurately measuring a biological signal by using a wristwatch-type measurement module. After a band of the wristwatch-type measurement module is tightened to wear on a user's wrist, the band is further tightened to make the wristwatch-type measurement module closely contact the user's wrist. An operation mode of the wristwatch-type measurement module closely contacting the user's wrist is switched from a normal mode to a measurement mode, and a user's biological signal is measured from the user's wrist through the wristwatch-type measurement module in the measurement mode. The user's biological signal is then displayed through the wristwatch-type measurement module.

### PRIORITY

This application claims priority under 35 U.S.C. §119(a) to a Korean Patent Application filed in the Korean Intellectual Property Office on Apr. 2, 2012 and assigned Serial No. 10-2012-0033790, the entire disclosure of which is incorporated herein by reference.

### BACKGROUND OF THE INVENTION

1. Field of the Invention

The present invention relates generally to an apparatus and method for measuring a biological signal, and more particularly, to an apparatus and method for easily and accurately measuring a biological signal by using a wristwatch-type measurement module.

2. Description of the Related Art

With the recent increased interest in healthcare and fitness, various apparatuses for scientifically managing health and exercise and preventing diseases such as obesity, have been proposed. For example, recently, apparatuses of a pedometer type, which are based on an accelerometer sensor, have been proposed to measure the amount of exercise for exercise management by being worn on a body.

Apparatuses for measuring the amount of exercise typically measure the number of heartbeats having a high correlation with the amount of calorie consumption, and as an example of those apparatuses, an apparatus provides a chest belt using an electrocardiogram (EKG) is worn on the user's chest to measure the number of heartbeats, and the measured number of heartbeats is wirelessly transmitted to a wristwatch. As another example, an apparatus in the form of an arm band for measuring the number of heartbeats through an optical sensor on an inner forearm and an apparatus for measuring the number of heartbeats through an optical sensor in the form of a ring on the user's finger are also available.

However, as described above, since the current apparatuses for measuring the amount of exercise are worn on the user's chest, inner forearm, and finger to measure the number of heartbeats, the user may be uncomfortable wearing these apparatuses to measure the number of heartbeats, and find it difficult to measure the amount of exercise. In addition, as the current apparatuses for measuring the amount of exercise are worn on the user in the form of a band, when the amount of exercise is measured during exercise, the apparatuses cannot make close contact with the user due to athletic wear and the sweat generated due to exercise, such that the measured number of heartbeats is not accurate and thus an accurate amount of exercise cannot be measured.

Moreover, there has been no detailed scheme which allows a user who has a chronic disease, such as heart disease, blood pressure related disease, etc., to easily and accurately measure a biological state, such as the number of heartbeats and blood pressure, for healthcare as well as for exercise management through measurement of the amount of exercise. In other words, the apparatuses for measuring the biological state for the user's current healthcare measure a predetermined biological state, e.g., a single biological state such as the number of heartbeats, blood pressure, body fat, or the like.

The current apparatuses for measuring a biological state are inconvenient for users to measure their biological states. Moreover, users have difficulties in accurately applying these apparatuses to their bodies to measure their biological states and cannot accurately measure their body states due to inaccurate apparatus application.

Therefore, for stable healthcare and exercise management, and to easily and accurately measure the amount of exercise and biological state of the user, a scheme for measuring the amount of exercise and biological state of the user, i.e., the user's biological signal is needed.

### SUMMARY OF THE INVENTION

Therefore, the present invention has been made to solve the above-stated problems occurring in the prior art, and, the present invention provides an apparatus and method for measuring a user's biological signal to stably perform user's healthcare and health management.

The present invention also provides an apparatus and method for easily and accurately measuring the amount of exercise and biological state of a user.

The present invention also provides an apparatus and method for easily and accurately measuring a biological signal by using a wristwatch-type measurement module to stably perform healthcare and exercise management of a user.

According to an aspect of the present invention, there is provided an apparatus for measuring a biological signal. The apparatus includes a first tightening portion for tightening a band of a wristwatch-type measurement module to wear on a user's wrist, a second tightening portion for further tightening the band to make the wristwatch-type measurement module closely contact the user's wrist, a button unit for switching an operation mode of the wristwatch-type measurement module, a sensor unit for measuring a user's biological signal from the user's wrist which the wristwatch-type measurement module closely contacts, and a display unit for displaying the user's biological signal.

According to another aspect of the present invention, there is provided a method for measuring a biological signal by using a wristwatch-type measurement module. The method includes, after tightening a band of the wristwatch-type measurement module to wear on a user's wrist, further tightening the band to make the wristwatch-type measurement module closely contact the user's wrist, switching an operation mode of the wristwatch-type measurement module closely contacting the user's wrist from a normal mode to a measurement mode, measuring a user's biological signal from the user's wrist through the wristwatch-type measurement module in the measurement mode, and displaying the user's biological signal through the wristwatch-type measurement module.

### BRIEF DESCRIPTION OF THE DRAWINGS

The above and other aspects, features and advantages of the present invention will be more apparent from the following detailed description taken in conjunction with the accompanying drawings, in which:

FIGS. 1-3 are diagrams illustrating an apparatus for measuring a biological signal according to an embodiment of the present invention;

FIGS. 4-6 are diagrams illustrating describing an operating state of an apparatus for measuring a biological signal according to an embodiment of the present invention;

FIG. 7 is a diagram illustrating a wristwatch-type measurement module in an apparatus for measuring a biological signal according to an embodiment of the present invention;

FIGS. 8-10 are diagrams illustrating apparatuses for measuring a biological signal according to other embodiments of the present invention; and

FIG. 11 is a flowchart illustrating an operating procedure of an apparatus for measuring a biological signal according to an embodiment of the present invention.

### DETAILED DESCRIPTION OF EMBODIMENTS OF THE PRESENT INVENTION

Hereinafter, an apparatus and method for measuring a biological signal according to various embodiments of the present invention will be described in detail with reference to the accompanying drawings. In the following description, line thicknesses or component sizes illustrated in the drawings may be exaggerated for clarity and convenience of the description. Terminologies used below are defined considering functions in the present invention, and the terminologies can be changed according to a user, an operator's intention, or custom. Therefore, the definition must be drawn on the basis of contents over this specification.

The present invention provides an apparatus and method for measuring a biological signal by using a wristwatch-type measurement module. Although a wristwatch-type measurement module is described as one example of an embodiment of the present invention, a scheme for measuring a biological signal provided in the present invention may also be applied to measurement of a biological signal using another type of a module.

In addition, according to an embodiment of the present invention, to sufficiently satisfy users' needs in healthcare and fitness, an apparatus and method for measuring a biological signal is provided which allows a user to easily and accurately measure a user's biological signal to perform exercise healthcare, and physical management. Herein, according to an aspect of an embodiment of the present invention described below, by using a wristwatch-type measurement module, a user can easily and accurately measure a biological signal of another person as well as a biological signal of the user. For example, according to an embodiment of the present invention, by using the wristwatch-type measurement module, the amount of exercise of the user is easily and accurately measured during a user's exercise, and a biological state of the user is easily and accurately measured in the daily life and a particular situation of the user.

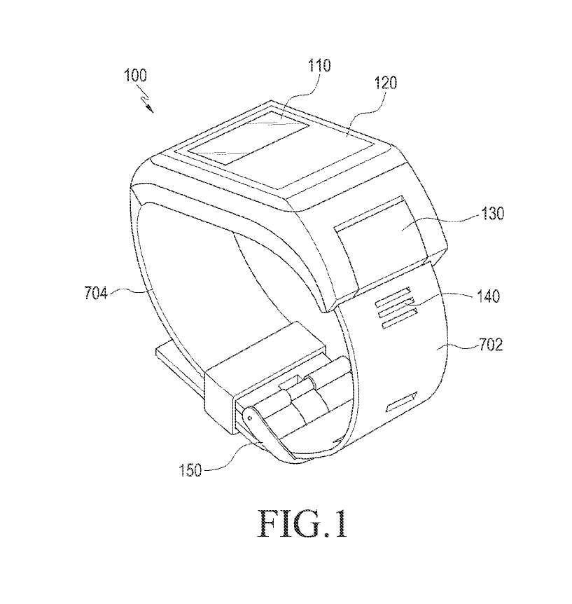

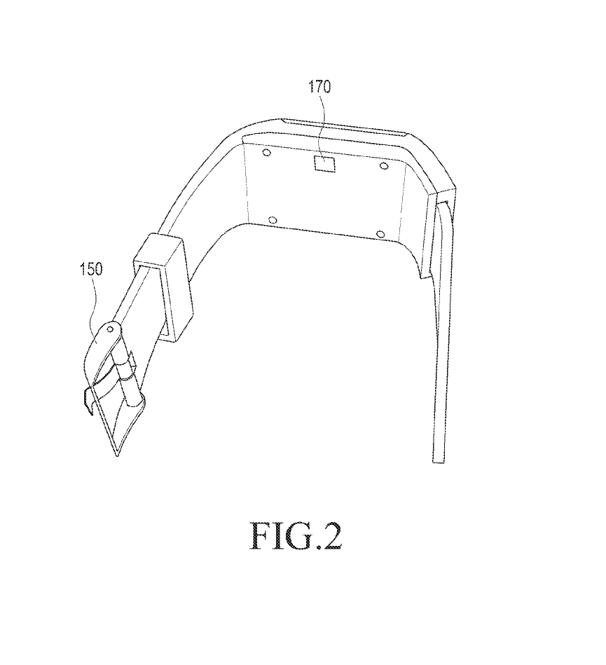

Herein, according to an embodiment of the present invention, to measure the amount of exercise or biological state of the user as described above, the user easily and accurately measures an electrocardiogram (EKG), the number of heartbeats, blood pressure, a blood oxygen saturation level, a heart rhythm, the number of breaths, blood sugar, a body temperature, etc., by using the wristwatch-type measurement module, thus measuring the biological signal of the user, and by using the measured biological signal of the user, the user's exercise, healthcare, and physical management can be stably performed. With reference to FIGS. 1-3, an apparatus for measuring a biological signal by using a wristwatch-type measurement module according to an embodiment of the present invention will be described in more detail.

FIGS. 1-3 are diagrams illustrating an apparatus for measuring a biological signal according to an embodiment of the present invention.

Referring to FIGS. 1-3, the biological signal measuring apparatus includes a first tightening portion 150 which causes a wristwatch-type measurement module 100, i.e., the biological signal measuring apparatus, to be worn on a user's wrist through bands 702 and 704, a second tightening portion 130 which tightens the bands 702 and 704, tightened by the first tightening portion 150, more tightly for measurement of the user's biological signal, a button unit 120 which switches the wristwatch-type measurement module 100 to a mode for measuring the user's biological signal, a sensor unit 170 which measures the user's biological signal, and a display unit 110 for displaying the user's biological signal measured by the sensor unit 170.

The first tightening portion 150 wraps and tightens the user's wrist with the bands 702 and 704, such that the wristwatch-type measurement module 100 capable of measuring the amount of exercise or biological state of the user, i.e., the biological signal measuring apparatus is worn on the user's wrist for user's exercise, healthcare, and physical management. That is, the first tightening portion 150 causes the wristwatch-type measurement module 100 to be worn on the user's wrist, and the user uses the wristwatch-type measurement module 100 worn on the wrist through the first tightening portion 150 as a regular wristwatch for checking the time when the user does not desire to measure the amount of exercise or biological state.

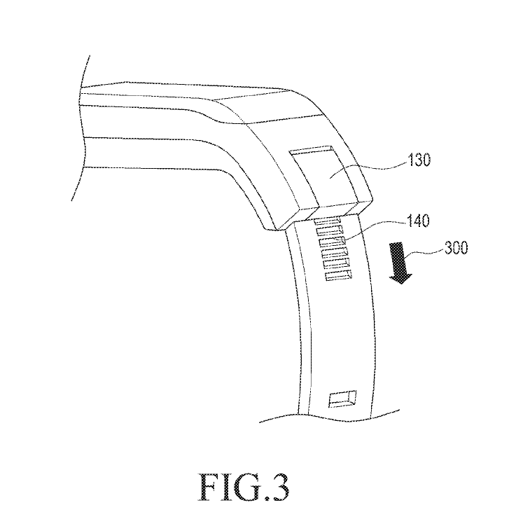

The second tightening portion 130, as mentioned above, further tightens the bands 702 and 704, which wraps the user's wrist, of the wristwatch-type measurement module 100 worn through the first tightening portion 150, when the user measures the amount of exercise or biological state. That is, the second tightening portion 130 further tightens the bands 702 and 704 of the wristwatch-type measurement module 100, making the wristwatch-type measurement module 100 make closer contact with the user's wrist. When the user measures the amount of exercise or biological state of the user, as illustrated in FIG. 3, the second tightening portion 130 is moved on predetermined saw-toothed grooves 140 formed in the bands 702 and 704 in a first direction 300, thus further tightening the bands 702 and 704 wrapping the user's wrist. When the user uses the wristwatch-type measurement module 100 as a regular wrist watch for checking the time after measuring the amount of exercise or biological state, the second tightening portion 130 is moved on the saw-toothed grooves 140 formed in the bands 702 and 704 in a direction reverse to the first direction 300, thus loosening the bands 702 and 704 wrapping the user's wrist. A more detailed description of the operation of the second tightening portion 130 which further tightens the bands 702 and 704 wrapping the user's wrist when the user measures the amount of exercise or biological state will be provided below and is thus omitted at this time.

The button unit 120, as mentioned previously, switches an operation mode of the wristwatch-type measurement module 100 from a normal mode to a measurement mode, so that the wristwatch-type measurement module 100 measures the amount of exercise or biological state of the user, that is, the user's biological signal when the user desires to measure the amount of exercise or biological state during the use of the wristwatch-type measurement module 100 as a regular wrist watch for checking the time. The button unit 120 also switches the operation mode of the wristwatch-type measurement module 100 from the measurement mode to the normal mode to use the wristwatch-type measurement module 100 as the regular wrist watch for checking time after measurement of the amount of exercise or biological state of the user. In other words, the user, when measuring the amount of exercise or biological state through the wristwatch-type measurement module 100, operates the button unit 120 to switch the operation mode of the wristwatch-type measurement module 100 from the normal mode to the measurement mode, and when using the wristwatch-type measurement module 100 as the regular wrist watch for checking the time, operates the button unit 120 to switch the operation mode of the wristwatch-type measurement module 100 from the measurement mode to the normal mode.

The sensor unit 170, as described above, measures the amount of exercise or biological state of the user when the operation mode of the wristwatch-type measurement module 100 is switched from the normal mode to the measurement mode through the button unit 120 and the user measures the amount of exercise or biological state. Herein, the sensor unit 170 measures an EKG or Photoplethysmography (PPG) signal, i.e., the user's biological signal. The sensor unit 170 measures, as the biological signal of the user, the number of heartbeats, blood pressure, a blood oxygen saturation level, a heart rhythm, the number of breaths, blood sugar, a body temperature of the user, etc. The sensor unit 170 also accurately measures the user's biological signal as the wristwatch-type measurement module 100 makes closer contact with the user's wrist by the second tightening portion 130 as described above.

The sensor unit 170 outputs the measured biological signal of the user through the display unit 110, and if the operation mode of the wristwatch-type measurement module 100 is switched from the measurement mode to the normal mode through the button unit 120 after measurement of the user's biological signal, then the sensor unit 170 ceases measurement of the user's biological signal.

In the normal mode of the wristwatch-type measurement module 100, the display unit 110 provides the current time or the like to the user, in the measurement mode of the wristwatch-type measurement module 100, the display unit 110 provides the user's biological signal measured by the sensor unit 170 to the user. Herein, the display unit 110, although not illustrated in detail, includes a transmitter (not shown) to transmit the user's biological signal measured in the measurement mode to a server which collectively manages user's exercise, healthcare, and physical management, e.g., a hospital server, a sports center server, or the like.

Herein, the biological signal measuring apparatus notifies the button unit 120 that the operation mode of the wristwatch-type measurement module 100 can be switched from the normal mode to the measurement mode when the wristwatch-type measurement module 100 makes closer contact with the user's wrist through tightening of the second tightening portion 120, such that the user is notified through the display unit 110 and the button unit 120 to switch the operation mode of the wristwatch-type measurement module 100 to the measurement mode by using the button unit 120. The biological signal measuring apparatus also notifies the user through the display unit 110 and the button unit 120 that the sensor unit 170 normally measures the user's biological signal in the measurement mode, and displays the measured user's biological signal through the display unit 110 and transmits the measured user's biological signal through the transmitter to the server which collectively manages user's exercise, healthcare, and physical management. The biological signal measuring apparatus, upon completing measurement of the user's biological signal in the measurement mode, notifies the user through the display unit 110 and the button unit 120 that measurement of the biological signal has been completed, that is, the operation mode of the wristwatch-type measurement module 100 can be switched to the normal mode.

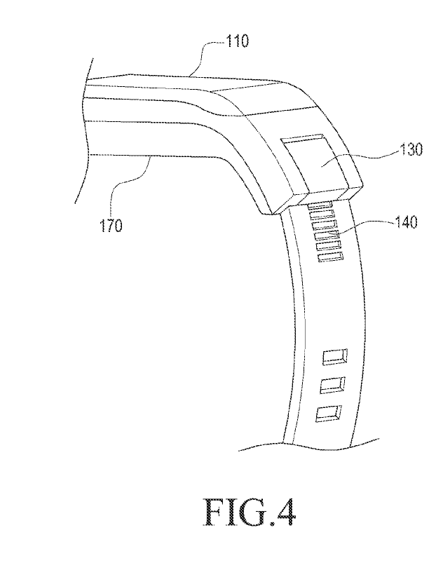

As such, the biological signal measuring apparatus according to an embodiment of the present invention allows the user to easily measure the biological signal of the user through the wristwatch-type measurement module 100, that is, to easily measure the biological signal of the user by wearing the wristwatch-type measurement module 100 on the user's wrist through the first tightening portion 150, and accurately measures the user's biological signal by making the wristwatch-type measurement module 100 closely contact the user's wrist through the second tightening portion 130. A more detailed description regarding the operation of the second tightening portion 130 in the wristwatch-type measurement module 100 according to an embodiment of the present invention is made with reference to FIGS. 4-6.

FIGS. 4-6 are diagrams illustrating describing an operating state of the apparatus for measuring a biological signal according to an embodiment of the present invention.

Referring to FIGS. 4-6, in the biological signal measuring apparatus, when the wristwatch-type measurement module 100 is used as a regular wrist watch for checking the time in the normal mode as illustrated in FIG. 4, the sensor unit 170 is turned off and the display unit 110 provides the current time to the user. To measure the user's biological signal by using the wristwatch-type measurement module 100 in this state, as described previously, the operation mode of the wristwatch-type measurement module 100 is switched from the normal mode to the measurement mode and then the second tightening portion 130 is moved on the saw-toothed grooves 140 in the first direction 300, thus making the wristwatch-type measurement module 100 closely contact the user's wrist.

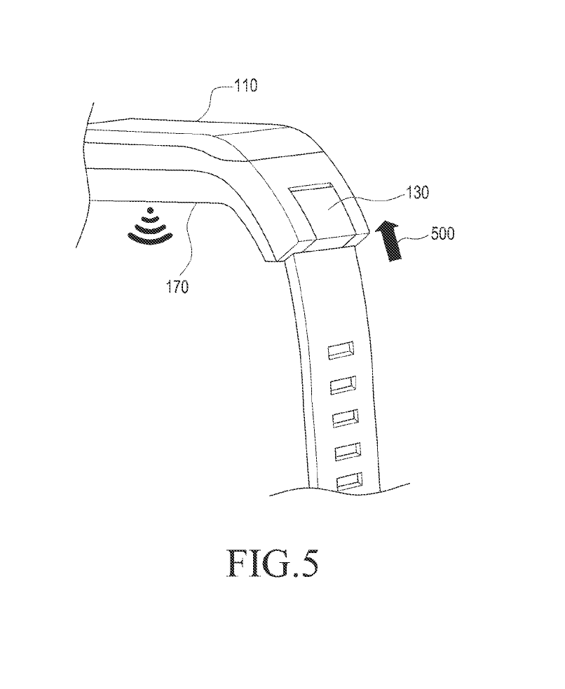

That is, as illustrated in FIG. 5, the biological signal measuring apparatus tightens the bands 702 and 704 through the second tightening portion 130 to make the wristwatch-type measurement module 100 closely contact the user's wrist, switches the operation mode of the wristwatch-type measurement module 100 to the measurement mode to turn on the sensor unit 170, and measures the user's biological signal through the turned-on sensor unit 170 in the measurement mode.

Herein, when the wristwatch-type measurement module 100 makes closer contact with the user's wrist through the second tightening portion 130, then the user is notified through the display unit 110 and the button unit 120 that the operation mode of the wristwatch-type measurement module 100 can be switched to the measurement mode, and the operation mode of the wristwatch-type measurement module 100 is switched to the measurement mode through the button unit 120. The sensor unit 170 measures, as the user's biological signal, an EKG or PPG signal, that is, the number of heartbeats, blood pressure, a blood oxygen saturation level, a heart rhythm, the number of breaths, blood sugar, a body temperature of the user, etc. The biological signal measuring apparatus provides the user's biological signal measured through the sensor unit 170 to the user through the display unit 110, and transmits the measured user's biological signal through the transmitter (not shown) to a server which collectively manages user's exercise, healthcare, and physical management.

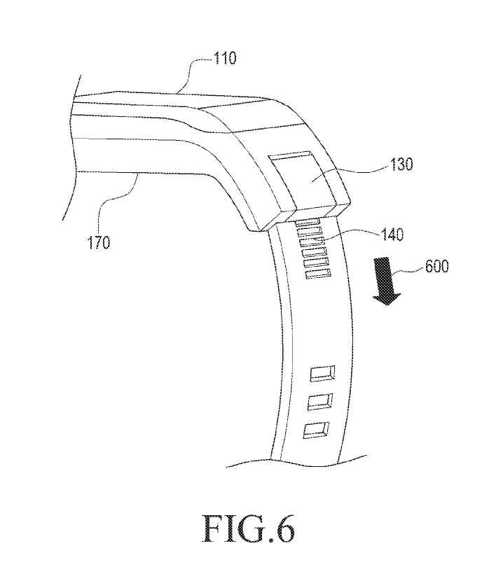

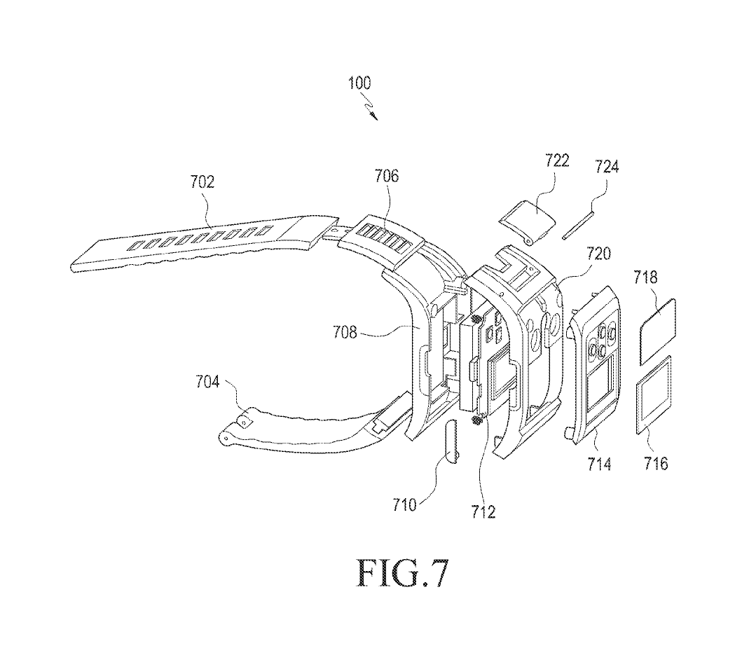

When the wristwatch-type measurement module 100 is used as the regular wrist watch for checking the time in the normal mode after measurement of the user's biological signal, the biological signal measuring apparatus moves the second tightening portion 130 on the saw-toothed grooves 140 in a second direction 500 to loosen the wristwatch-type measurement module 100 on the user's wrist. That is, in the biological signal measuring apparatus, as illustrated in FIG. 6, the second tightening portion 130 loosens the bands 702 and 704, and the operation mode of the wristwatch-type measurement module 100 is switched to the normal mode, such that the time is provided to the user through the display unit 110 in the normal mode. With reference to FIG. 7, a detailed description will be made regarding a structure of the biological signal measuring apparatus according to an embodiment of the present invention.

FIG. 7 is a diagram illustrating the structure of the wristwatch-type measurement module 100 in the biological signal measuring apparatus according to an embodiment of the present invention.

Referring to FIG. 7, as described above, the wristwatch-type measurement module 100 includes the first tightening portion 150, the second tightening portion 130, the button unit 120, the sensor unit 170, and the display unit (110 of FIGS. 1, 4, 5, and 6), and the first tightening portion 150 couples the first band 702 and the second band 704, which wrap the user's wrist, to each other, thus causing the wristwatch-type measurement module 100 to be worn on the user's wrist.

The second tightening portion 130 includes a band lock 706 in which saw-toothed grooves are formed, a lever 722 which moves on the band lock 706 to further tighten or loose the bands 702 and 704, and a moving pin 724 which allows the lever 722 to move on the band lock 706.

As mentioned previously, the button unit 120 notifies the user that the operation mode of the wristwatch-type measurement module 100 can be switched, and includes a button sheet 718 for switching the operation mode of the wristwatch-type measurement module 100.

The display unit (110 of FIGS. 1, 4, 5, and 6) notifies the user that the operation mode of the wristwatch-type measurement module 100 can be switched, and includes a Liquid Crystal Display (LCD) window 716 which provides a user's biological signal measured through the sensor unit 170 to the user by displaying the measured user's biological signal.

Herein, the wristwatch-type measurement module 100 includes a coupling module 714 for coupling the button sheet 718 and the LCD window 716 to the wristwatch-type measurement module 100, a circuit module 712 for driving the wristwatch-type measurement module 100, a cover 720 for coupling the circuit module 712 to the wristwatch-type measurement module 100, and an auxiliary cover 710 for protecting an external memory connection port. The structure of the wristwatch-type measurement module 100, i.e., the biological signal measuring apparatus, has already been descried in detail and thus a detailed description thereof will not be provided.

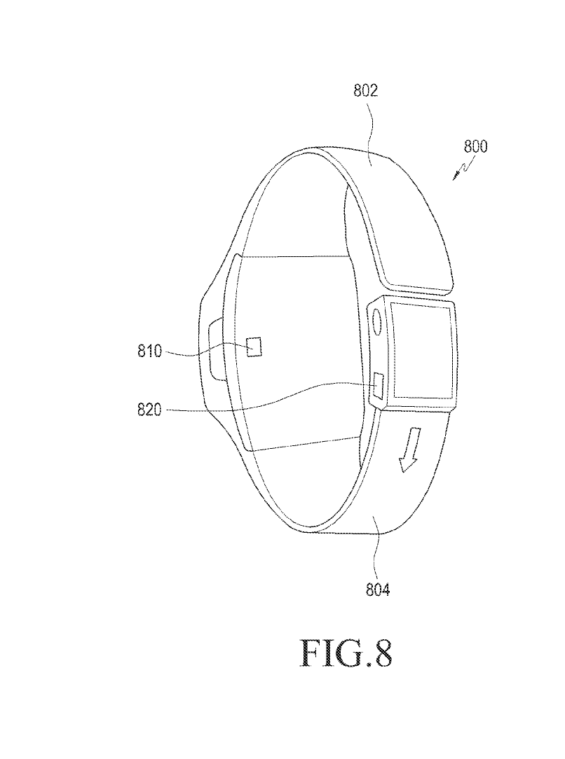

Hereinafter, an apparatus for measuring a biological signal according to another embodiment of the present invention will be described in detail with reference to FIGS. 8-10.

FIGS. 8-10 are diagrams illustrating apparatuses for measuring a biological signal according to other embodiments of the present invention. First, as illustrated in FIG. 8, the biological signal measuring apparatus further tightens bands 802 and 804 of a wristwatch-type measurement module 800 through a second tightening portion 820 in the form of a lever, causing the wristwatch-type measurement module 800 to closely contact the user's wrist, and measures a user's biological signal through a sensor unit 810 in a measurement mode of the closely contacting wristwatch-type measurement module 800.

Herein, the biological signal measuring apparatus easily measures the user's biological signal through the wristwatch-type measurement module 800, and moves the second tightening portion 820 on the bands 802 and 804 of the wristwatch-type measurement module 800 in a predetermined direction to further tightens the bands 802 and 804 wrapping the user's wrist, thus making the wristwatch-type measurement module 800 closely contact the user's wrist, such that the user's biological signal can be more accurately measured. Moreover, if the operation mode of the wristwatch-type measurement module 800 is switched to the normal mode after completion of measurement of the biological signal, then the second tightening portion 820 in the form of a lever is moved on the bands 802 and 804 in a direction reverse to the predetermined direction, thus loosening the previously tightened bands 802 and 804. The wristwatch-type measurement module 800 including the second tightening portion 820 in the form of a lever is similar to the wristwatch-type measurement module 100 described with reference to FIGS. 1-7, except for the second tightening portion 820, and thus will not be described in detail.

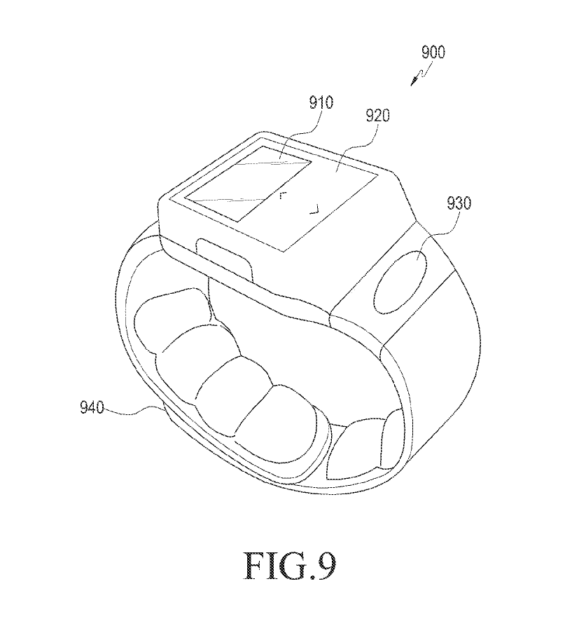

As illustrated in FIG. 9, a biological signal measuring apparatus further tightens a band of a wristwatch-type measurement module 900 through second tightening portions 930 and 940 of an air injection type to make the wristwatch-type measurement module 900 closely contact the user's wrist, switches the operation mode of the closely contacting wristwatch-type measurement module 900 to the measurement mode through mode switch of a button unit 920 to measure the user's biological signal through the sensor unit 170 in the measurement mode, and provides the measured user's biological signal to the user through a display unit 910.

Herein, the biological signal measuring apparatus easily measures the user's biological signal through the wristwatch-type measurement module 900 and makes the wristwatch-type measurement module 900 closely contact the user's wrist through the second tightening portions 930 and 940 of the air injection type, thus more accurately measuring the user's biological signal. The second tightening portions 930 and 940 of the air injection type include a tightening button 930 and an air band 940. If the tightening button 930 is pushed to cause the wristwatch-type measurement module 900 closely contact the user's wrist for biological signal measurement in the measurement mode, then air is injected into the air band 940 which exists in an inner side of the band wrapping the user's wrist and thus the band is tightened, such that the wristwatch-type measurement module 900 makes closer contact with the user's wrist, thereby more accurately measuring the user's biological signal. If the operation mode of the wristwatch-type measurement module 900 is switched to the normal mode after completion of the measurement of the biological signal, then the tightening button 930 is pushed to exhaust the air injected into the air band 940, thus loosening the previously tightened band. The wristwatch-type measurement module 900 including the second tightening portions 930 and 940 of the air injection type is similar to the wristwatch-type measurement module 100 described with reference to FIGS. 1-7, except for the second tightening portions 930 and 940, and thus will not be described in detail.

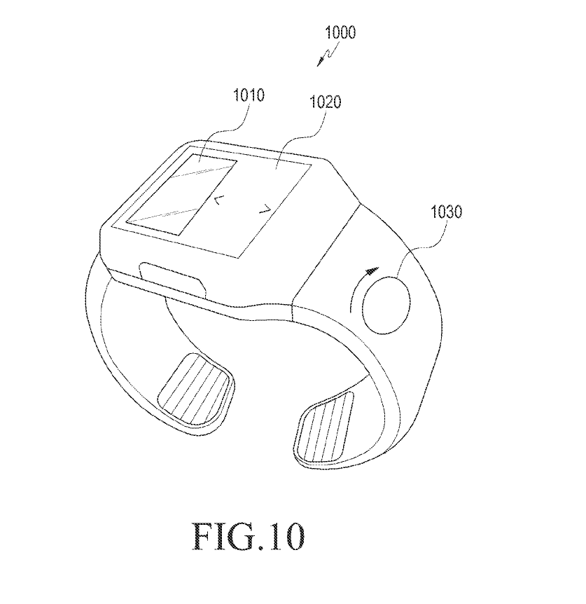

As illustrated in FIG. 10, a biological signal measuring apparatus further tightens a band of a wristwatch-type measurement module 1000 through a second tightening portion 1030 of a Boa mechanism type to make the wristwatch-type measurement module 1000 closely contact the user's wrist, switches the operation mode of the closely contacting wristwatch-type measurement module 1000 to the measurement mode through mode switch of a button unit 1020 to measure the user's biological signal through the sensor unit 170 in the measurement mode, and provides the measured user's biological signal to the user through a display unit 1010.

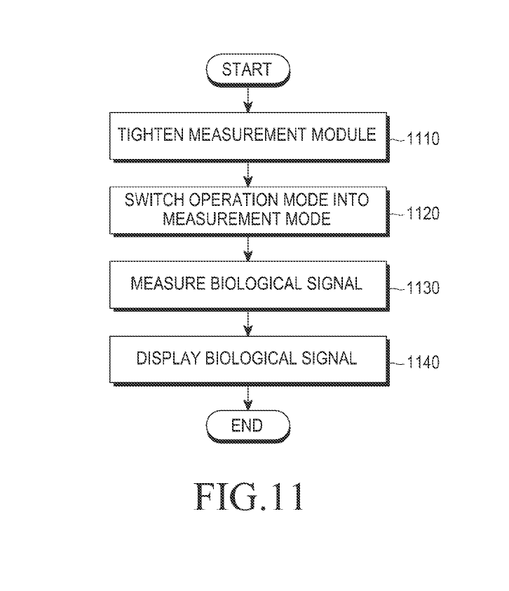

Herein, the biological signal measuring apparatus measures the user's biological signal through the wristwatch-type measurement module 1000 and makes the wristwatch-type measurement module 1000 closely contact the user's wrist through the second tightening portion 1030 of the Boa mechanism type, thus more accurately measuring the user's biological signal. The second tightening portion 1030 is a bore button, such that the second tightening portion 1030 of the Boa mechanism type is pushed and in this state, is rotated in a predetermined direction to more tighten the band wrapping the user's wrist, thus making the wristwatch-type measurement module 1000 closely contact the user's wrist and thereby more accurately measuring the user's biological signal. If the operation mode of the wristwatch-type measurement module 1000 is switched to the normal mode after completion of measurement of the biological signal, then the second tightening portion 1030 of the Boa mechanism type is pulled to loosen the previously tightened band. The wristwatch-type measurement module 1000 including the second tightening portion 1030 of the Boa mechanism type is similar to the wristwatch-type measurement module 100 described with reference to FIGS. 1-7, except for the second tightening portion 1030 of the Boa mechanism type, and thus will not be described in detail. Hereinafter, an operation of measuring a user's biological signal by using a wristwatch-type measurement module according to an embodiment of the present invention will be described with reference to FIG. 11.

FIG. 11 is a flowchart of an operating procedure of an apparatus for measuring a biological signal according to an embodiment of the present invention.

Referring to FIG. 11, when the wristwatch-type measurement module is worn on the user's wrist through the first tightening portion and the user checks the time in the normal mode, to measure the user's biological signal, the biological signal measuring apparatus tightens the band wrapping the user's wrist through the second tightening portion. That is, the biological signal measuring apparatus tightens the wristwatch-type measurement module, thus making the wristwatch-type measurement module closely contact the user's wrist in step 1110.

In step 1120, the biological signal measuring apparatus switches the operation mode of the wristwatch-type measurement module from the general mode to the measurement mode through the button unit.

In step 1130, the biological signal measuring apparatus measures the user's biological signal through the sensor unit. Herein, the sensor unit measures, as the user's biological signal, an EKG or PPG signal, that is, the number of heartbeats, blood pressure, a blood oxygen saturation level, a heart rhythm, the number of breaths, blood sugar, a body temperature of the user, etc.

In step 1140, the biological signal measuring apparatus provides the measured biological signal to the user by displaying it to the user through the display unit, and transmits the measured biological signal through the transmitter to the server which collectively manages user's exercise, healthcare, and physical management.

As such, the biological signal measuring apparatus according to an embodiment of the present invention easily measures the user's biological signal through the wristwatch-type measurement module, and makes the wristwatch-type measurement module make closer contact with the user's wrist, thus accurately measuring the user's biological signal.

As is apparent from the foregoing description, by measuring the user's biological signal with the wristwatch-type measurement module, the user can easily and accurately measure the amount of exercise and biological state of the user, thereby stably managing user's healthcare and exercise.

While the present invention has been described with reference to certain embodiments thereof, it will be understood by those skilled in the art that various modifications in form and detail can be made therein without departing from the spirit and scope of the present invention as defined by the appended claims and their equivalents.

What is claimed is:

1. An apparatus for measuring a biological signal, the apparatus comprising:
a first tightening portion for tightening a band of a wristwatch-type measurement module to wear on a user's wrist;
a second tightening portion for further tightening the band to make the wristwatch-type measurement module closely contact the user's wrist;
a button unit for switching an operation mode of the wristwatch-type measurement module;
a sensor unit for measuring a user's biological signal from the user's wrist which the wristwatch-type measurement module closely contacts; and
a display unit for displaying the user's biological signal.

2. The apparatus of claim 1, wherein the button unit switches the operation mode of the wristwatch-type measurement module from a normal mode to a measurement mode when the wristwatch-type measurement module closely contacts the user's wrist.

3. The apparatus of claim 2, wherein the sensor unit measures a user's Electrocardiogram (EKG) or Photoplethysmography (PPG) signal from the user's wrist when in the measurement mode.

4. The apparatus of claim 2, wherein the sensor unit measures at least one of a number of heartbeats, blood pressure, a blood oxygen saturation level, a heart rhythm, a number of breaths, blood sugar, and a body temperature of the user from the user's wrist when in the measurement mode.

5. The apparatus of claim 1, further comprising a transmitter for transmitting the user's biological signal to a server for exercise management and healthcare of the user.

6. The apparatus of claim 2, wherein the button unit switches the operation mode of the wristwatch-type measurement module from the measurement mode to the normal mode when the sensor unit completes the measurement of the user's biological signal.

7. The apparatus of claim 5, wherein when in the normal mode, the second tightening portion loosens the band of the wristwatch-type measurement module, and
the wristwatch-type measurement module displays the time on the display unit.

8. The apparatus of claim 1, wherein the second tightening portion is moved on predetermined saw-toothed grooves formed in the band in a first direction to further tighten the band, and is moved on the saw-toothed grooves in a direction opposite to the first direction to loosen the band.

9. The apparatus of claim 1, wherein the second tightening portion moves a lever on the band in a first direction to further tighten the band, and moves the lever in a direction reverse to the first direction to loosen the band.

10. The apparatus of claim 1, wherein the second tightening portion injects air into an inner side of the band to further tighten the band and exhausts the air injected into the inner side of the band to loosen the band.

11. The apparatus of claim 1, wherein the second tightening portion rotates a Boa mechanism button in a pushed state in a first direction on the band to further tighten the band and pulls the Boa mechanism button to loosen the band.

12. A method for measuring a biological signal by using a wristwatch-type measurement module, the method comprising:
after tightening a band of the wristwatch-type measurement module to wear on a user's wrist, further tightening the band to make the wristwatch-type measurement module closely contact the user's wrist;
switching an operation mode of the wristwatch-type measurement module closely contacting the user's wrist from a normal mode to a measurement mode;
measuring a user's biological signal from the user's wrist through the wristwatch-type measurement module in the measurement mode; and
displaying the user's biological signal through the wristwatch-type measurement module.

13. The method of claim 12, wherein measuring the user's biological signal comprises measuring a user's Electrocardiogram (EKG) or Photoplethysmography (PPG) signal from the user's wrist.

14. The method of claim 12, wherein measuring the user's biological signal comprises measuring at least one of a number of heartbeats, blood pressure, a blood oxygen saturation level, a heart rhythm, a number of breaths, blood sugar, and a body temperature of the user from the user's wrist.

15. The method of claim 12, further comprising transmitting the user's biological signal to a server for exercise management and healthcare of the user through the wristwatch-type measurement module.

16. The method of claim 12, wherein switching the operation mode comprises switching the operation mode of the wristwatch-type measurement module from the measurement mode to the normal mode when the wristwatch-type measurement module completes the measurement of the user's biological signal.

17. The method of claim 16, further comprising:
loosening the band in the normal mode; and
displaying the time through the wristwatch-type measurement module.
